(19) United States
(12) Patent Application Publication
(10) Pub. No.: US 2026/0335489 A1
(43) Pub. Date: Oct. 29, 2026

(54) DEVICE FOR DISPENSING VOLATILE SUBSTANCES, IN PARTICULAR FRAGRANCES AND/OR ACTIVE SUBSTANCES

(71) Applicant: CTR, LDA, Samora Correia (PT)

(72) Inventors: Harold AUGIER, Zhongshan City (CN); Luo MIN, Zhongshan (CN)

(21) Appl. No.: 19/115,380

(22) PCT Filed: Sep. 26, 2022
(86) PCT No.: PCT/EP2022/076649

§ 371 Date: Mar. 26, 2025

(51) Int. Cl. A61L 9/03

(52) U.S. Cl. CPC A61L 9/037; A61L 2209/11

(57) ABSTRACT

A device for dispensing, in particular for vaporizing, volatile substances, in particular fragrances and/or active substances, has a housing with at least one discharge opening for a substance hot-air flow generated in the housing. The container with the substance to be dispersed has a wick projecting from the container with a wick end that forms a substance delivery area at the outflow opening. An electric heating device has a controller for setting different heating powers. In a booster operation, a predeterminable booster heating power is settable, with which a booster heat quantity for an associated booster substance release rate is transferable from a heating element, preferably to the wick end. For a basic operation, a predeterminable basic heating power is set which is lower than the booster heating power and where a comparatively lower basic heat quantity is transferred from the heating element for a lower basic substance release rate.

The invention refers to a device for dispensing, in particular for vaporizing, volatile substances, in particular fragrances and/or active substances, according to the preamble of claim 1, and to a method according to the preamble of claim 14.

Such devices for dispensing, in particular for vaporizing, volatile substances, in particular fragrances and/or active substances, are generally known in various designs and regularly comprise a housing with at least one outflow opening for a substance hot air flow generated in the housing as a plume. A container with the substance to be dispensed is inserted into the housing. The container has a wick which protrudes from the container with a wick end forming a substance delivery region in the region of the at least one outflow opening. The device also has an electrical heating device with an electrical heating element arranged in the region of the wick end. Thus, when the heating device is activated, heat is applied to the free wick end in order to be able to quickly release the substance accumulating in the free wick end to the environment or to evaporate it. Such a structure is known, for example, from WO 98/58692 A1. In order to be able to variably adjust the degree of evaporation and thus the evaporation output, the container together with the wick is mounted in a height-adjustable manner in the housing of the device, whereby the relative position of the wick to the heating device can be changed and the heating output of the heating device fixed to the housing remains unchangeably the same. Such a mechanically adjustable mounting of the container is relatively complex and cost-intensive to manufacture and can also lead to the following operating problems:

To achieve the strongest possible effect, a user of the device can set the highest substance release rate at the smallest distance between the heating device and the wick end. This can have the disadvantage that the free wick end dries out and is dehumidified to a great extent due to the high heat effect, whereby the capillary structure in the wick end can be severely damaged by sticking and/or clumping, so that the substance release rate is considerably reduced and, in extreme cases, no more substance evaporates and a partially still filled container has to be replaced. If, on the other hand, the heating device is set in the other extreme position with maximum distance to the end of the wick, the substance release may be so low that damage to the wick is avoided in this way, but a desired effect can only be achieved insufficiently. If necessary, a user can find out an intermediate position of the heating device by his own tests, which represents a compromise between the two positions explained above, but this requires time-consuming tests and adjustments.

With the control device described above, it is advantageously possible to set a high substance release rate for rapid action and rapid distribution of a plume in a room after activation of the device. Disadvantageously, such a setting can be reset by a user to a lower continuous substance release rate after a waiting period.

It is therefore an object of the invention to propose a relatively simple and inexpensive device with which, after activation and/or if necessary, a rapid distribution of the plume with the substances contained can be achieved.

This object is solved with the features of the independent patent claims. Advantageous embodiments are the subject of the related subclaims.

According to claim 1 it is provided a device for dispensing, in particular for vaporizing, volatile substances, in particular fragrances and/or active substances, comprising
a housing which has at least one discharge opening for a substance hot-air flow generated in the housing,
a container which is connectable to the housing, preferably insertable into the housing, and which has the substance to be dispensed, the container having a wick projecting from the container with a wick end forming a substance delivery area in the region of the at least one outflow opening, and
an electric heating device which has at least one heating element, preferably in the region of the wick end.

According to the invention, the electrical heating device has a control device which is suitable and designed to set different heating powers, preferably in such a way that
for a booster operation, a predeterminable booster heating power is settable, with which a booster heat quantity for an associated booster substance release rate is transferable from the at least one heating element, preferably to the wick end, and
for a basic operation, a predeterminable basic heating power is settable which is lower than the booster heating power and in which a comparatively lower basic heat quantity is transferable from the at least one heating element for an associated comparatively lower basic substance release rate, preferably to the wick end.

This advantageously achieves that after an activation of the device and thus an activation of the control device, in particular after an initial activation, a plume of high intensity at a high substance release rate is generated and with a fast effective function. After such an initial booster function, the control device sets, preferably automatically, a lower basic heating power, when after a certain time the substance effect has spread in the room.

In a more specific embodiment, the electrical heating device is an electrical resistance heater with a heat-generating overall resistance that can be activated for heat generation, wherein the electrical heating device can be connected to an energy source for energy supply and can be supplied with a predetermined electrical voltage, wherein it is preferably provided that the heat-generating overall resistance is formed by the at least one heating element. Such a structure is characterized by a high degree of functional reliability and is also easy to manufacture.

In addition, a structure in which the control device is suitable and designed to change the overall heat-generating resistance of the heating device in order to set the heating power of the heating device is particularly advantageous. Alternatively or additionally, the control device can be suitable and designed to change the electrical voltage in order to adjust the heating power of the heating device.

For reliable operation of the device, it is also advantageous if the at least one heating element comprises a heating body made of a thermally conductive material and at least one electrical resistor element thermally coupled to the heating body, preferably at least partially integrated in the heating body. A single heating body may also include two or optionally more resistor elements to form a plurality of interconnected heating elements.

In a preferred concrete embodiment, the heating device has two electrical heating elements connected in parallel in an electrical heating circuit, each with at least one resistor element, wherein the control device simply and inexpensively has a thermal switch, preferably in the housing in the vicinity of the heating device and/or in a parallel branch, with which one of the two heating elements can be switched on and off. This is preferably done in such a way that the thermal switch is closed in the cold state and thus, after an initial activation of the device, both parallel-connected heating elements are activated in a booster operation in the heating circuit, and/or that the thermal switch opens after reaching its switching temperature and switches off one of the two heating elements, the temperature still rising or overshooting due to the thermal inertia of the arrangement and then falling, preferably falling to a constant value for a basic operation. In other words, the thermal switch is closed after an initial activation of the device in the cold state, so that both heating elements connected in parallel are activated in a booster operation in the heating circuit. As a result, the wick end is rapidly heated and the thermal switch is also heated such that it opens after reaching its switching temperature and turns off one of the two heating elements, with the temperature still rising or overshooting due to the thermal inertia of the arrangement and then dropping to a constant value for a base operation.

Preferably, PTC resistor elements, i.e. resistor elements with a positive temperature coefficient, are used as resistor elements for particularly functionally reliable operation.

Alternatively or additionally, the resistor elements of the two heating elements can have or form different resistors with different heating power, whereby in booster mode, with the thermal switch closed, a heating element with lower heating power (low PTC) is or is connected to a heating element with higher heating power (high PTC).

The respective resistance of the resistor elements of the heating elements and/or the relative positions of the heating elements with respect to the thermal switch are preferably suitable and formed,
in that the thermoswitch remains open after its switching temperature has been reached by sufficiently further heating and the device can continue to be operated at a constant base temperature and associated base substance release rate,
or
in that, after the second heating element has been switched off, the heat transfer to the thermoswitch is reduced in such a way that its switching temperature falls below again after a cooling time, so that the thermoswitch closes again for a further booster operation and thus the heating power or the temperature and correspondingly the substance release rate change periodically due to the thermal inertia, preferably in the manner of a sawtooth curve and/or in the region resp. range above the basic substance release rate.

By dimensioning the resistance of the resistor elements in the heating elements and/or by the relative fixed positioning of the thermal switch with respect to the heating elements, the thermal switch can thus remain open after an initial activation of the device and after a booster procedure with a further sufficient heating, so that the device remains activated with a constant base temperature at the wick end and associated base substance release rate. Alternatively, with appropriate dimensioning of the heating elements and arrangement of the thermal switch, after the second heating element has been switched off, the heat transfer to the thermal switch can be so low that its switching temperature falls below again after a cooling time, so that the thermal switch is reset for further booster operation and closes, whereby the heating power or the temperature and correspondingly the substance release rate change periodically due to the thermal inertia, preferably in the manner of a sawtooth curve and/or in the range resp. region above the base substance release rate. In principle, the two alternative modes of operation are possible here, but it is advantageous in any case if an initial booster operation takes place after an initial activation of the device.

In a particularly preferred embodiment, the device can be designed as a plug-in component and have plug-in contacts projecting from the housing, which are suitable and designed to be plugged into a socket, preferably on the room wall side, for supplying the heating device with electrical energy and for holding the device. The electrical heating device is also preferably suitable and designed to carry out an initial activation of the device by plugging the plug-in component into a socket and the associated application of energy.

In a further advantageous further development of the device, the thermal switch can be assigned a booster button which is arranged on the housing and can be actuated externally and by means of which the thermal switch, when it is open in basic operation, can be closed again manually as required, in particular for a further booster operation after an initial booster operation, and the second heating element can be switched on again for a further booster time and a correspondingly increased heating power. This means, for example, that after an initial booster operation, when the thermal switch is open in basic operation, the thermal switch can be closed again manually for a further booster operation if required, whereby the second heating element can be switched on again for a further booster time, in particular because of the thermal inertia of the arrangement, for a greater heating power.

Advantageously, the thermal switch is an R.TCO (resettable thermal switch with transparent conducting oxides), in particular in the embodiment without booster button, or alternatively a bimetallic switch, in particular in the embodiment with booster button.

As the inventor's tests have shown, undesirable condensation and vortex formation as well as a reduction in the outflow velocity can be reduced in particular if the outflow opening is arranged, preferably approximately centrally, on an upper side of the housing, preferably on a cover shell of the housing. Particularly in connection with a structure comprising a container with wick as described above, it is furthermore advantageous if the upper side, preferably the cover shell, of the housing is located in this case above the heating device and/or the outflow opening is located above the wick end, as seen in the vertical axis direction.

When we speak above of a cover shell, we are referring here to a component forming the upper side of the housing, which can be a separate housing component, e.g. as a separate shell part placed on the housing, or of course can also be formed and/or shaped as a single piece of material with the housing. In the functional position or viewed in the vertical axis direction, the upper side or cover shell is preferably aligned approximately horizontally or extends in a horizontal plane.

In its functional position, the device, preferably as a plug-in component in the plugged-in state in a socket, preferably on the room wall side, is to be aligned with its housing in such a way that the wick associated with the heating device, preferably the wick surrounded by the heating device, stands approximately vertically in the housing and the outflow opening in the housing is located at a distance above it.

Furthermore, the object is solved with respect to the method for dispensing, in particular for vaporizing, volatile substances, in particular fragrances and/or active substances, with the features of claim 14. The advantages resulting from the method according to the invention correspond analogously to those of the device according to the invention, so that reference is made to the explanations previously given in order to avoid repetition.

The advantageous embodiments and further developments of the invention explained above and/or reproduced in the subclaims can be used individually or also in any combination with one another-except, for example, in the cases of clear dependencies or incompatible alternatives.

The invention and its advantageous embodiments and further developments are explained in more detail below with reference only to exemplary and schematic drawings.

### The Drawings Show

FIG. 1 a perspective view of an exemplary embodiment according to the invention of a device for dispensing, in particular for vaporizing, volatile substances, in particular fragrances and/or active substances, with a housing, an inserted container and plug contacts for connection to a socket,

FIG. 2 a view corresponding to FIG. 1, without the housing, which is only indicated by dashed lines, with the internal structure with a heating device and a wick,

FIG. 3 a top perspective view of the heating device,

FIG. 4 a schematic circuit diagram of a first embodiment with two electrical heating elements connected in parallel and a thermal switch formed by an R.TCO,

FIG. 5 a temperature curve in the area of the wick end in a circuit according to FIG. 4 in a first operating mode,

FIG. 6 a temperature curve at the wick end in a circuit according to FIG. 4 in a second operating mode,

FIG. 7 a view corresponding to FIG. 1 according to a further development to a second embodiment with a booster button,

FIG. 8 a view corresponding to FIG. 2 with a heating device with booster button,

FIG. 9 a top view of a heating device according to FIG. 3 with a modification according to the second embodiment,

FIG. 10 a schematic representation of a circuit diagram corresponding to FIG. 4 with a thermal switch as a bimetal switch with additional manual booster function,

FIG. 11 a temperature curve at the wick end using the circuit shown in FIG. 10 after initial activation of the device and a subsequent manual booster.

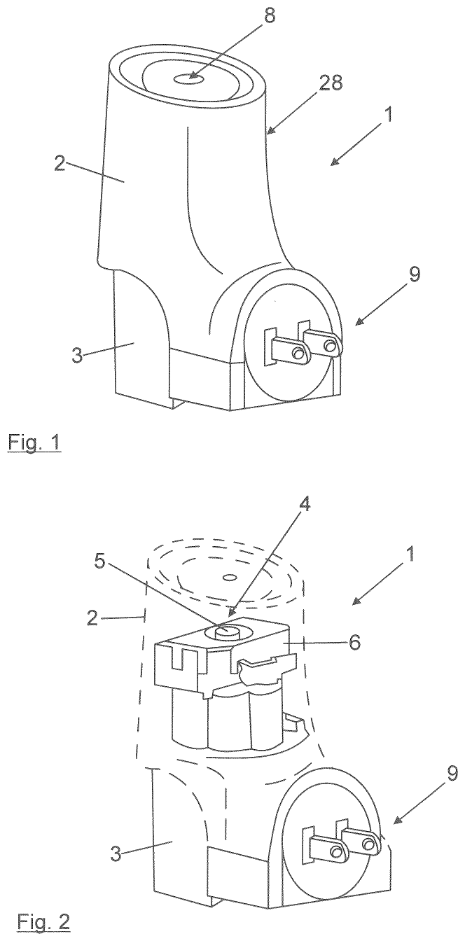

FIG. 1 shows a device 1 for vaporizing volatile substances, in particular fragrances and/or active substances, with a housing 2 in which a container 3 containing the substance to be dispensed is inserted. From the internal structure of the device 1 shown in FIG. 2, it is apparent that a wick 4 received in the container protrudes from the container with a wick end 5 through a heating device 6. After activation of the heating device 6, heat is transferred to the wick end and thus a substance hot air flow is generated which forms a plume (not shown). An outlet opening 8 is provided in the housing 2 above the wick end 5.

The device 1 is, merely exemplary here, L-shaped in its basic design and forms a plug-in component. In its functional position, the housing 2 is aligned in such a way that the wick 4 surrounded by the heating device 6 is approximately vertical in the housing 2, namely with the outflow opening 8 located at a distance above it.

On a lower L-leg of the housing 2, plug contacts 9 protrude from the housing 2, which can be plugged into a socket 10 on the room wall side (see FIG. 14) for supplying the heating device 6 with electrical energy and for holding the device 1 on the associated room wall.

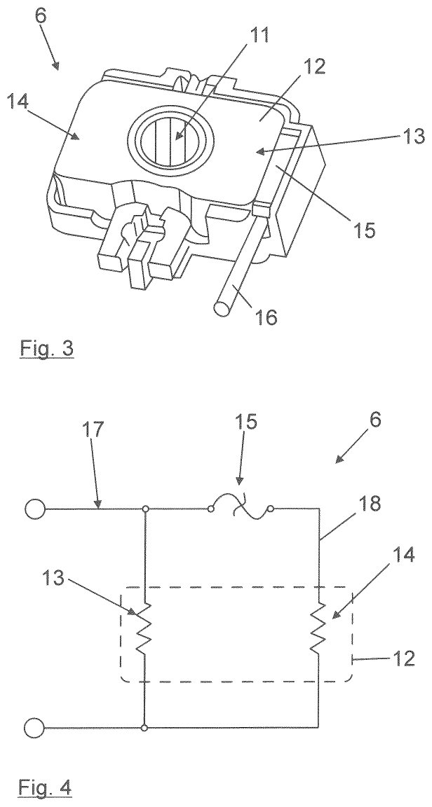

FIG. 3 shows an electric heater 6 with a central wick-receiving opening 11 in a heater body 12. The heater body 12 consists of a thermally conductive material in which two electrical heating elements 13, 14 with (not visible) integrated electric resistor elements are contained. FIG. 3 also schematically shows a thermoswitch as R.TCO 15 and an electric cable 16 for the electric power supply.

The electrical circuit in connection with the heating function are shown in FIGS. 4 to 6:

From the schematic circuit diagram of FIG. 4 it can be seen that the heating element 13 and the heating element 14 are connected in parallel in an electrical heating circuit 17, whereby after activation of the heating circuit 17 the heating element 13 is permanently switched on and the heating element 14 can be, according to its temperature rise, switched off and, if necessary, switched on again with the thermoswitch 15 in its supply line 18.

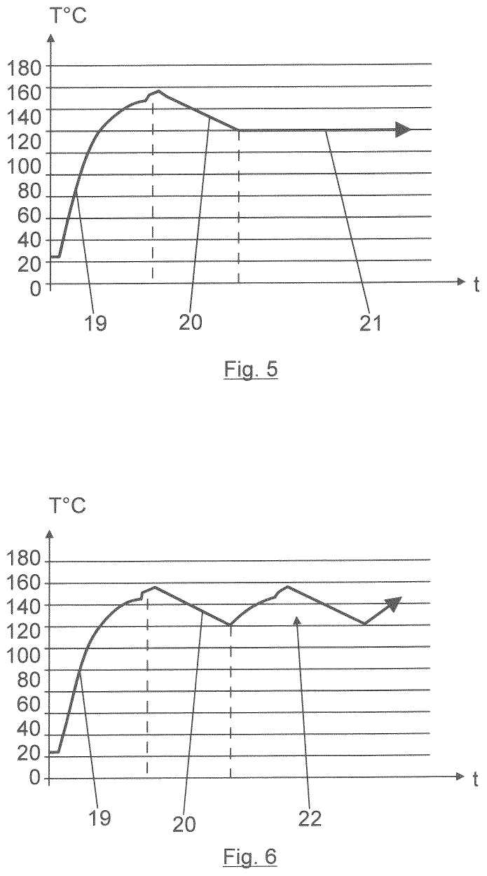

For this purpose, the thermal switch 15 is arranged as part of a control device in the housing 3 in the area of the heating device 6. The initial activation of the device 1 and thus of the heating device 6 takes place when the plug-in contacts 9 are plugged into a socket 10 by the associated electrical power supply. During this process, the thermal switch 15 is still cold and closed and the heating device operates with the maximum possible heating power with the heating elements 13, 14 connected in parallel in an initial booster mode. FIG. 5 shows the associated temperature curve in the region of the wick end 5, with the temperature rising relatively quickly in accordance with a first curve section 19. As soon as the switching temperature of the thermal switch 15 is exceeded, for example at 110 °C, the thermal switch 15 opens and the heating element 14 is switched off. Due to the thermal inertia of the arrangement, the temperature rises further to up to about 150 °C in accordance with FIG. 5, in which the values are selected merely by way of example, and then drops to a temperature value of 120 °C, which remains constant in the third curve section 21, due to the reduced heating power of the heating device 6 in the second curve section 20. In the example shown here, this value is above the switching temperature of the thermal switch 15, so that the thermal switch 15 remains open for further continuous operation without the heating element 14.

The resistor elements in the heating elements 13 and 14 are here preferably PTC resistor elements, i.e. resistor elements with a positive temperature coefficient, whereby the resistor elements of the two heating elements 13, 14 have different resistances with different heating power. In booster mode, the heating element 14 with low heating power (low PTC) is connected to the heating element 13 with higher heating power (high PTC) when the thermal switch 15 is closed, and in a further continuous mode corresponding to the third curve section 21 in FIG. 5, the heating element 14 (low PTC) remains switched off. The resettable thermoswitch 15 as R.TCO is arranged close to the heating element with higher heating power (high PTC), as can be seen in particular in FIG. 3.

Depending on the dimensions of the heating elements 13, 14, the relative distances to the thermal switch 15 and the selection of the switching temperature of the thermal switch 15, a periodic temperature curve as shown in FIG. 6 is also possible if, after the falling temperature curve in the wide curve section 20, the thermal switch 15 switches back below its switching temperature and closes again. Then the second heating element 14 is switched on again in a further booster operation and a periodically varying temperature with a correspondingly periodically varying substance release rate is set in accordance with the curve profile 22.

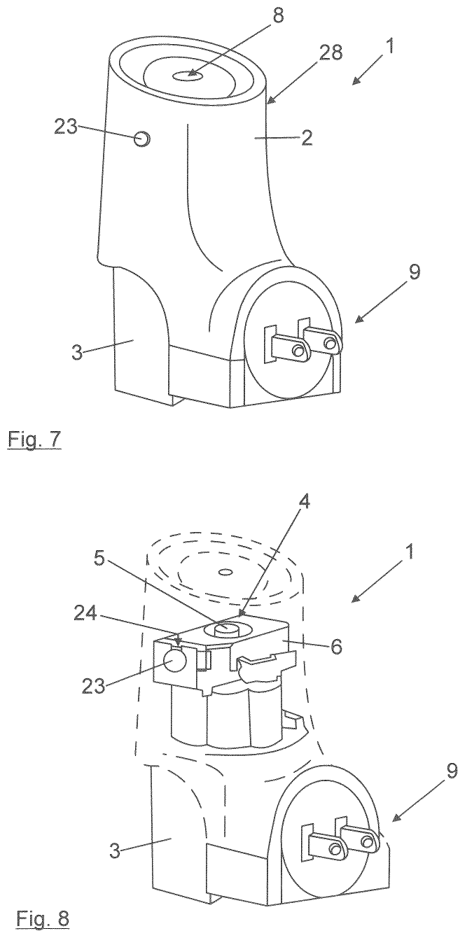

A second embodiment corresponding to FIGS. 7 to 11 largely corresponds to the first embodiment with further development by means of a booster button 23 which protrudes from the housing 2 and which is associated with a thermal switch 15, which is designed here, for example, as a bimetal switch 24.

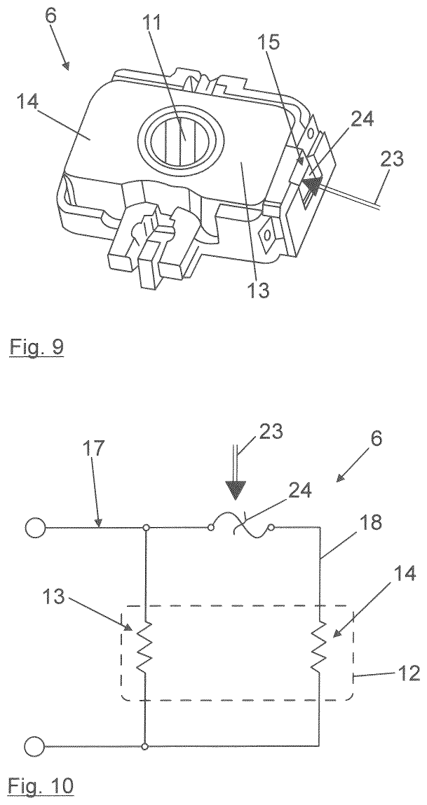

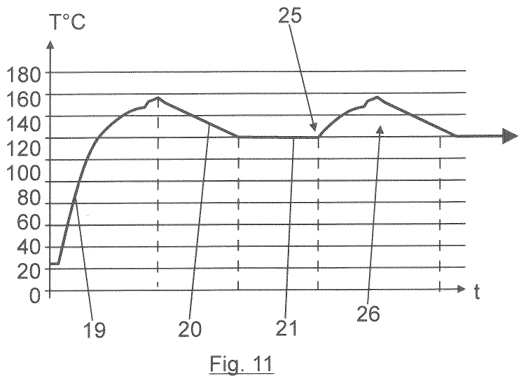

In FIGS. 9 and 10, the booster button is shown schematically as an arrow 23.

The circuit diagram of the second embodiment according to FIG. 10 corresponds largely to the circuit diagram of the first embodiment according to FIG. 4 and is merely supplemented by the booster button 23, with which the bimetal switch 24 can be reset manually to its closed position with activation of the heating element 14 individually if required.

Thus, the temperature curve shown in FIG. 11 can be realized. After initial activation of the device 1, without actuation of the booster button 23, a curve is produced corresponding to the three curve sections 19, 20, 21 from FIG. 5. At time 25, the booster button 23 is then actuated and, by switching on the heating element 14, a temperature increase is triggered with booster operation, as shown in curve section 26, which then changes back to a constant temperature curve corresponding to curve section 21.

### LIST OF REFERENCE SIGNS

1 Device
2 Housing
3 Container
4 Wick
5 Wick end
6 Heating device
8 Outflow opening
9 Plug contacts
10 Socket
11 Wick-receiving opening
12 Heater body
13 Heating element (high PTC)
14 Heating element (low PTC)
15 Thermoswitch (R.TCO)
16 Electrical cables
17 Heating circuit
18 Cable
19 First curve section
20 Second curve section
21 Third curve section
22 Curve section
23 Booster button
24 Bimetal switch
25 Time
26 Curve section
28 Cover shell

1-14. (canceled)

15. A device for dispensing a volatile substance, the device comprising:
a housing formed with at least one discharge opening for a substance hot-air flow generated in the housing;
a container to be connected to said housing and containing the substance to be dispensed, said container having a wick projecting from said container with a wick end forming a substance delivery area in the region of said at least one outflow opening;
an electric heating device having at least one heating element; and
said electric heating device having a controller configured to set different heating powers, wherein:
in a booster operation, a predeterminable booster heating power is set, with which a booster heat quantity for an associated booster substance release rate is transferred from said at least one heating element to said wick; and
in a basic operation, a predeterminable basic heating power is set which is lower than the booster heating power and in which a comparatively lower basic heat quantity is transferred from the at least one heating element to said wick for an associated comparatively lower basic substance release rate.

16. The device according to claim 15, wherein said container is formed for insertion into said housing.

17. The device according to claim 15, wherein said heating element is disposed at said wick end and the heat in the heating operations (booster, basic) are transferred to the wick end.

18. The device according to claim 15, wherein said electrical heating device is an electrical resistance heater with a heat-generating total resistance which is activatable for heat generation, wherein said electrical heating device is connectable to an energy source for energy supply and is suppliable with a predetermined electrical voltage.

19. The device according to claim 18, wherein the heat-generating total resistance is formed by the at least one heating element.

20. The device according to claim 18, wherein at least one of the following is true:
said controller is configured for changing the heat-generating total resistance of said heating device in order to set the heating power of the heating device;
or
said controller is configured for changing the electrical voltage in order to set the heating power of said heating device.

21. The device according to claim 15, wherein said at least one heating element comprises a heating body made of a thermally conductive material and at least one electrical resistor element thermally coupled to said heating body.

22. The device according to claim 21, wherein said electrical resistor is at least partially integrated in said heating body.

23. The device according to claim 15, wherein:
said heating device has two electrical heating elements which are connected in parallel in an electrical heating circuit and each have at least one resistor element; and
said controller has a thermal switch in at least one of a vicinity of said heating device or in a parallel branch, and said thermal switch is configured to selectively switch one of said two heating elements on or off in such a way that:
said thermal switch is closed in a cold state and thus, after an initial activation of the device, both heating elements connected in parallel are activated in a booster mode in the heating circuit; and
said thermal switch opens after reaching a switching temperature and switches off one of said two heating elements, the temperature still rising or overshooting due to a thermal inertia and then falling towards a constant value for basic operation.

24. The device according to claim 23, wherein at least one of the following is true:
each of said resistor elements is formed by a positive temperature coefficient (PTC) resistor element; or
said resistor elements of said two heating elements have respectively different resistances with different heating power and, in the booster mode, with said thermal switch closed, a heating element with lower heating power (Low PTC) is connected to a heating element with higher heating power (High PTC).

25. The device according to claim 23, wherein at least one of the resistances of said resistor elements of said heating elements or relative positions of said heating elements with respect to said thermal switch is configured so that:
said thermal switch remains open after a switching temperature thereof has been reached by sufficiently further heating and the device can continue to be operated with a constant base temperature and associated base substance release rate;
or
after switching off a second heating element, the heat transfer to said thermal switch is reduced in such a way that the switching temperature thereof falls below again after a cooling time, so that said thermal switch closes again for a further booster operation and thus the heating power or the temperature and the substance release rate change periodically due to the thermal inertia.

26. The device according to claim 25, wherein a temperature and a substance release rate change in a sawtooth curve in a region above the basic substance release rate.

27. The device according to claim 23, wherein:
the device is a plug-in component with plug-in contacts which project out of said housing and are to be plugged into a socket for supplying said heating device with electrical energy and for holding the device; and
said electrical heating device is configured to carry out an initial activation of the device by plugging said plug-in contacts into a socket and an associated application of energy.

28. The device according to claim 23, wherein said thermal switch is a resettable thermal switch with transparent conducting oxides (R.TCO).

29. The device according to claim 23, wherein said thermal switch is assigned a booster button disposed on said housing and actuatable externally for switching said thermal switch, when said thermal switch is open in basic operation, for manually closable said thermal switch as required, for triggering a further booster operation after an initial booster operation, wherein a second heating element is switched on again for a further booster time and a correspondingly increased heating power.

30. The device according to claim 29, wherein said thermal switch is a bimetallic switch.

31. The device according to claim 15, wherein said outflow opening is arranged approximately centrally on an upper side of said housing.

32. The device according to claim 31, wherein said upper side of said housing is formed by a cover shell that is located above said heating device, and said outflow opening is formed above said wick end of said wick in a vertical axis direction.

33. The device according to claim 15, wherein the device, in a functional position as a plug-in component in a plugged-in state in a socket, is aligned with said housing in such a way that said wick associated with said heating device stands approximately vertically in said housing and said outflow opening in said housing is located at a distance above it.

34. The device according to claim 33, wherein said wick surrounded by the heating device.

35. A method, comprising:
providing the device according to claim 15; and
vaporizing and dispensing volatile substances via the outflow opening.
